(19) United States
(12) Patent Application Publication
(10) Pub. No.: US 2008/0215011 A1
(43) Pub. Date: Sep. 4, 2008

(54) ENEMA DEVICE

(75) Inventor: Kenneth L. Beechie, University Place, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: IRON FIRE INNOVATIONS, LLC, University Place, WA (US)

(21) Appl. No.: 12/040,545

(22) Filed: Feb. 29, 2008

Related U.S. Application Data
(60) Provisional application No. 60/892,465, filed on Mar. 1, 2007.

(51) Int. Cl. A61M 3/02

(52) U.S. Cl. 604/187; 604/279

(57) ABSTRACT

An enema device for use with a source of enema fluid is provided. The enema device includes a hollow elongated body having first and second ends an interior extending between the first and second ends, a feed portion formed at the first end of the hollow elongated body, and an applicator portion disposed at the second end of the elongated body. The feed portion is in fluid communication with the interior of the hollow elongated body and is adapted to be placed into fluid communication with a source of enema fluid. The applicator portion defines a discharge opening in communication with the interior of the hollow elongated body.

### CROSS-REFERENCE TO RELATED APPLICATION

This application claims the benefit of U.S. Provisional Application No. 60/892,465, filed on Mar. 1, 2007, the disclosure of which is hereby expressly incorporated by reference.

### BACKGROUND

An enema is a common medical procedure whereby fluid is injected into the rectum of a patient in order to induce bowel movement. The need for such a procedure typically arises in patients suffering from certain physical ailments in which voluntary bowel control is impaired.

Medical equipment currently exists in the art for administering an enema to patients in need of this medical procedure. At least one type of equipment consists of an enema squeeze bottle filled with the fluid intended to induce bowel movement, which is capped by a short applicator nozzle to be inserted into the patient's rectum. The applicator nozzle of this type of conventional enema application device often causes discomfort and irritation when being inserted. Therefore, it is desired to have an enema device that safely and effectively administers the enema to a patient without causing discomfort.

Moreover, enemas are often administered to a patient at home when the need for medical assistance does not necessitate a doctor or another health care assistant. However, it is often difficult for the patient to administer the enema to him or herself since the applicator nozzle must be inserted into such a small, sensitive area. Moreover, it is difficult for the patient to administer the fluid while steadily holding the enema in the required area. Often the patient is assisted by another individual; however, assistance may not always be available, if, for instance, the patient lives alone. Thus, there is also a need for an enema device that can be effectively self-administered.

### SUMMARY

An enema device for use with a source of enema fluid is provided. The enema device includes a hollow elongated body having first and second ends an interior extending between the first and second ends, a feed portion formed at the first end of the hollow elongated body, and an applicator portion disposed at the second end of the elongated body. The feed portion is in fluid communication with the interior of the hollow elongated body and is adapted to be placed into fluid communication with a source of enema fluid. The applicator portion defines a discharge opening in communication with the interior of the hollow elongated body.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of the present disclosure will become more readily appreciated by reference to the following detailed description, when taken in conjunction with the accompanying drawings, wherein:

FIG. 1 is an environmental view of an enema device constructed in accordance with one embodiment of the present disclosure, wherein the enema device is shown in use with a pre-packaged enema bottle;

FIG. 2 is an isometric view of the enema device of FIG. 1;

FIG. 3 is a cross-sectional isometric view of the enema device of FIG. 2;

FIG. 4 is a first alternate embodiment of a portion of the enema device of FIG. 2;

FIG. 5 is a second alternate embodiment of a portion of the enema device of FIG. 2; and

FIG. 6 is a partial cross-sectional view of an alternate embodiment of an enema device, wherein the enema device is shown in use with a pre-packaged enema bottle.

### DETAILED DESCRIPTION

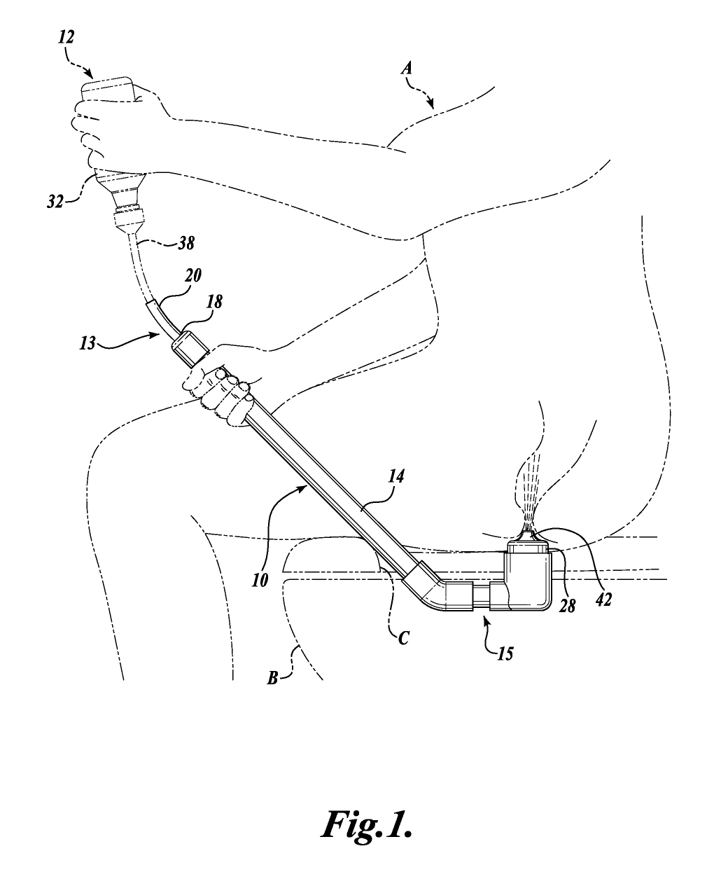

An enema device 10 shown in accordance with one embodiment of the present disclosure is best seen by referring to FIG. 1, wherein the enema device is being used by a patient A sitting on a toilet B. The enema device 10 is shown in use with a prepackaged disposable enema 12 that contains a suitable medicated fluid used for enemas. It should be appreciated that the enema device 10 may instead be used with any suitable enema, such as an enema bag or a bulb syringe. The enema device 10 may be used by a patient A to self-administer the enema when sitting on the toilet B; however, the patient A may instead be standing or sitting in any suitable position such that the enema device 10 may effectively be used.

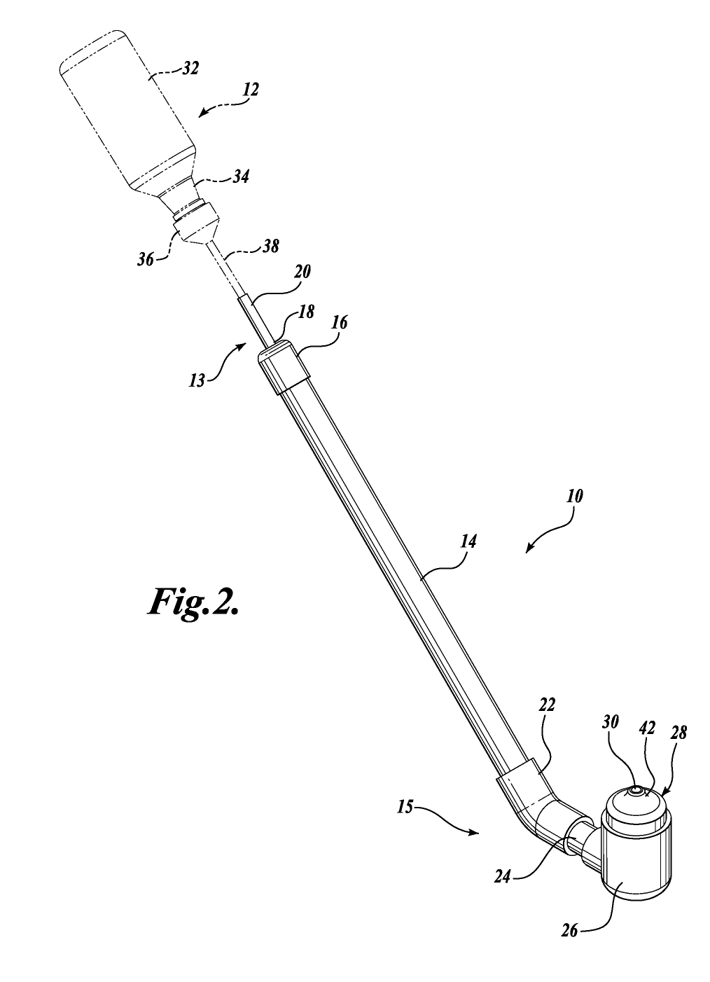

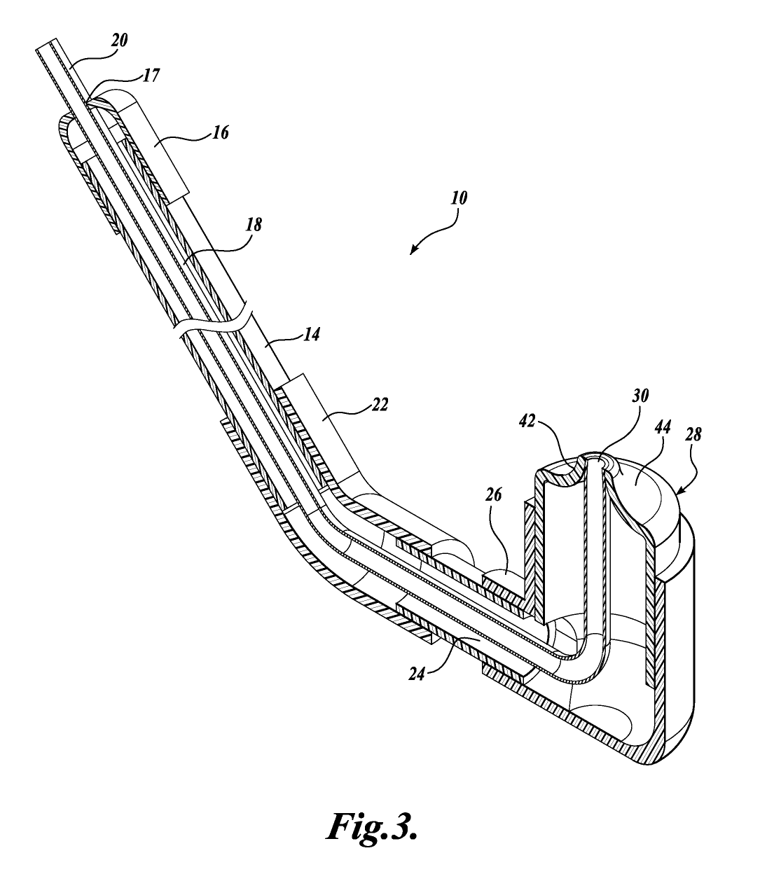

Referring to FIGS. 2 and 3, the enema device 10 includes a hollow, rigid elongated body 14 that acts as a lever against the toilet B or another suitable abutment device to brace the lower enema device 10 against the patient's buttocks (see FIG. 1). The elongated body 14 is preferably circular in cross section or any other suitable cross sectional shape. The elongated body 14 is substantially straight such that it acts as a suitable lever against the toilet B; however, it should be appreciated that the elongated body 14 may instead be suitably curved or contoured to position the enema device 10 against the front interior portion C of a toilet B or similar device. Moreover, an adjustable brace (not shown) may also be received on the elongated body 14 to help position and maintain the elongated body 14 against the front interior portion C of a toilet B. The elongated body 14 may also include a telescoping feature (not shown) that would allow the patient to lengthen or shorten the enema device 10 as needed. The elongated body 14 is preferably made of any suitable rigid material, such as polyvinyl chloride (PVC).

The enema device 10 includes a feed portion 13 disposed at the upper end of the elongated body 14 that is adapted to engage the prepackaged enema 12 and receive the medicated fluid from the prepackaged enema 12. The feed portion 13 includes a hollow cap 16 that is tightly received on the upper end of the elongated body 14 to enclose the upper end of the elongated body 14. The cap 16 includes a relatively small opening 17 in substantially the center of the cap that is adapted to receive a transport tube 18. The transport tube 18 is preferably made of a flexible material, such as rubber, and it extends throughout the length of the elongated body 14 and upwardly and outwardly from the cap 16 through the opening 17. The protruding portion of the transport tube 18 defines a feed tube portion 20 that is of a length and diameter to tightly receive, for instance, a nozzle 38 of the prepackaged enema 12 within the upper end of the feed tube portion 20.

It should be appreciated that the transport tube 18 may instead receive a separate feed tube (not shown) thereon that is adapted to receive the nozzle 38 of the prepackaged enema 12. The feed tube could be made of a flexible or semi-flexible material, such as rubber, and may be of a suitable length to act as an extension of the enema device 10 and facilitate easy insertion of the nozzle 38 within the feed tube at a desired distance from the enema device 10 or by another person.

In an alternative configuration, the transport tube 18 may not utilize a feed tube portion 20. Rather, a tapered opening may be formed in the cap 16 for receiving the nozzle 38 of the prepackaged enema 12. Such opening is tapered to receive nozzles 38 of various sizes. In this configuration, the tube 18 is in fluid flow communication with the tapered opening formed in the cap 16.

The enema device 10 further includes an applicator portion 15 disposed at the lower end of the elongated body 14 that is adapted to position the enema device 10 against the buttocks of the patient A for application of the enema. The applicator portion 15 includes a first elbow fitting 22 that is tightly received at one end on the lower end of the elongated body 14. The first elbow fitting 22 preferably includes only a slight bend such that an obtuse angle is formed between the elongated body 14 and the first elbow fitting 22. A sleeve fitting 24 is tightly received within the other end of the first elbow fitting 22, and a second elbow fitting 26 is tightly received on the sleeve fitting 24. The second elbow fitting 26 includes a substantially horizontal portion that is secured to the sleeve fitting 24 and a substantially vertical portion that defines a circular opening adapted to receive an end cap 28 therewithin.

The end cap 28 is circular in cross section and includes curved or contoured edges such that it is comfortably received against the patient's buttocks. The end cap 28 includes an end face 44 having a discharge opening 30 formed in substantially the center of the end face 44 that is adapted to receive the lower end of the transport tube 18 tightly therewithin. The transport tube 18 is positioned within the opening 30 such that the end of the tube 18 is substantially flush with the end face 44 of the end cap 28.

Preferably, the end cap 28 is contoured to help align the opening 30 against the patient's rectum and provide comfort to the patient A during use. Any suitable end cap shape may be used. As a non-limiting example, the end cap 28 may include a self-centering upward protrusion 42 formed around the opening 30, as shown in FIGS. 1-3. The self-centering upward protrusion 42 may be any suitable contoured shape to engage the opening of the rectum. For instance, the end face 44 may be slightly concave and extend upwardly toward the opening 30 to define the self-centering protrusion 42 and provide a soft, curved engagement surface. The self-centering protrusion 42 may instead be a small dome (not shown) formed on a substantially flat end face 44.

In the alternative configuration of cap 16 discussed above, the nozzle 38 of the enema bottle cap 36 is conveniently engaged within the tapered opening formed in cap 16 of the device feed portion 13. It will be appreciated that the cap 16 may be formed with a thicker or alternative configuration end portion than shown in FIG. 3, so as to provide sufficient length for the tapered opening formed therein. Once the nozzle 38 is engaged within such tapered opening, the prepackaged enema 12 is placed in secure fluid communication with the enema device 10.

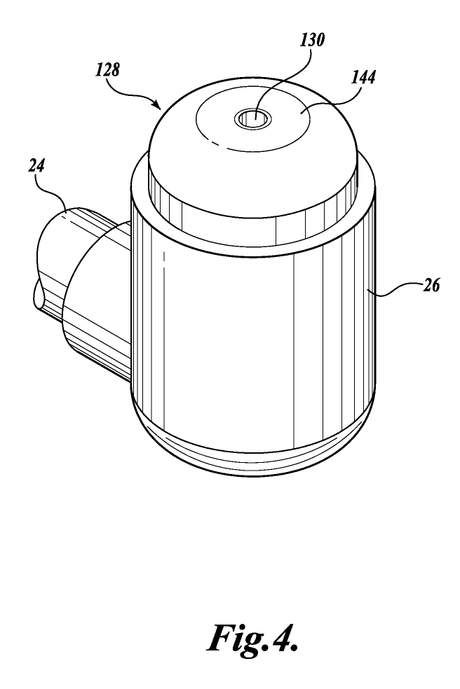

FIG. 4 depicts a first alternate embodiment of an end cap 128, wherein the end cap 128 includes a substantially flat end face 144 with an opening 130 formed therein. The end cap 128 includes a curved perimeter edge to provide a contoured engagement surface.

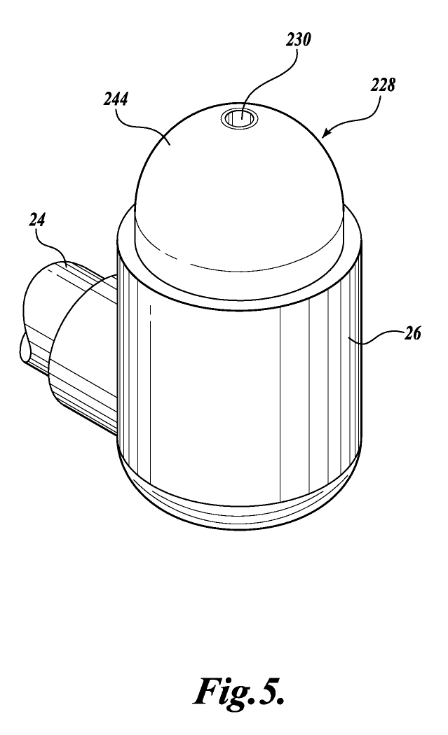

FIG. 5 depicts a second alternate embodiment of an end cap 228, wherein the end cap 228 includes a convex, dome surface 244 having a central opening 230 formed therein. It should be appreciated that any suitable contoured end cap shape may instead be used. Moreover, the end cap 28 may be any suitable cross-sectional shape, such as round, elliptical, etc.

Although the enema device 10 has been illustrated and described as having many different parts, it should be appreciated that the rigid components of the enema device 10 (i.e. the cap 16, elongated body 14, first elbow fitting 22, sleeve fitting 24, second elbow fitting 26, and end cap 28) may instead be formed together as one hollow piece formed by injection molding or another suitable method. Moreover, the enema device 10 may be any suitable shape such that it may effectively be used to administer an enema, as described below. Furthermore, the enema device 10 need not include a transport tube 18. Rather, the medicated fluid may instead be received and transported within the rigid components of the enema device 10.

Referring to FIG. 2, the feed portion 13 of the enema device 10 is adapted to receive the medicated fluid from the prepackaged disposable enema 12 or any other suitable enema. The prepackaged disposable enema 12 preferably includes an enema bottle 32 having a threaded bottle neck 34 that receives a twist-on threaded bottle cap 36. The enema bottle 32 is preferably prefilled with a medicated fluid that is well known in the art. The upper end of the bottle cap 36 includes an opening that is in communication with a nozzle 38 secured to the bottle cap 36. The nozzle 38 is adapted to be tightly received within the feed tube portion 20 of the transport tube 18 to place the prepackaged enema 12 into fluid communication with the enema device 10.

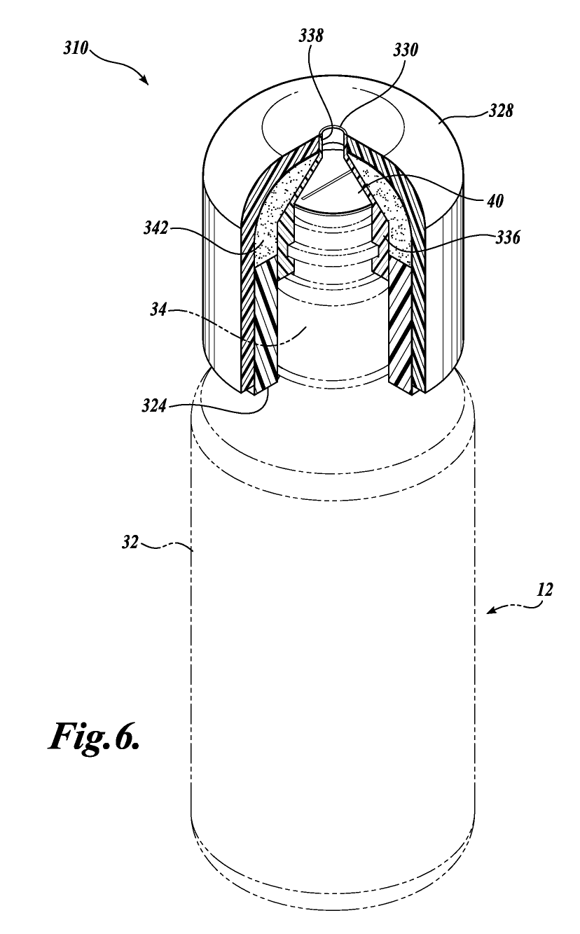

As can best be seen be referring to FIG. 6, the bottle cap 36 includes a one-way anti-reflux valve 40 that is positioned within the bottle cap 36 such that it encloses the opening in the bottle neck 34. The one-way valve 40 is preferably made from a non-porous flexible material and can be circular in shape, and is of a size which permits accommodation into the bottle cap 36. The one-way valve 40 prevents reflux of the medicated fluid back up into the bottle 32, thereby holding back any contaminants as well as helping to sustain the collapsibility of the bottle 32.

Referring to FIG. 1, the enema device 10 is most easily used when the patient is seated on a toilet B. To use the enema device 10, the patient A positions the applicator portion 15 beneath his or her buttocks such that a portion of the elongated body 14 and the feed portion 13 extend upwardly and outwardly from the toilet B in front of the patient A. The patient A then positions the end cap 28 properly against the rectum such that the self-centering protrusion 42 is received therein and the opening 30 is in fluid communication therewith.

The patient A thereafter pushes the top portion of the elongated body 14 outwardly to engage the elongated body 14 on the front interior portion C of the toilet B and pivot the elongated body 14 on the front interior portion C. The top portion of the elongated body 14 is pushed outwardly until the end cap 28 is firmly engaged against the rectum. While holding the enema device 10 in this position, the patient inserts the nozzle 38 of the prepackaged enema 12 into the feed tube 20. The patient A then squeezes the bottle 32 to force the medicated fluid out of the valve 40, through the nozzle 38, and into the transport tube 18. The fluid travels forcibly down through the transport tube 18 until it reaches the opening 30 in the end cap 28. The medicated fluid exits the opening 30 and projects upwardly into the patient's rectum. In this manner, the patient is able to administer an enema without the help of another person, and without inserting an uncomfortable tip into the rectum. However, it should be appreciated that the enema device 10 may instead be used with the assistance of a second person.

Now referring to FIG. 6, an alternate embodiment of an enema device 310 includes an end cap 328 that is adapted to be received onto the bottle neck 34 of a disposable enema bottle 32. The enema device 310 includes a sleeve fitting 324 that is adapted to be received over the lower portion of the bottle neck 34. An end cap 328, substantially similar in shape and size to the end cap 28 of the enema device 10 is tightly received over at least a portion of the sleeve fitting 324.

The end cap 328 and sleeve fitting 324 are adapted to receive a cap 336 and nozzle 338 substantially similar to the cap 36 and nozzle 38 that come with the prepackaged enema 12 except that the nozzle 338 is shorter in length. The end cap 328 includes an opening 330 that is adapted to receive at least a portion of the tip 338 such that the tip 338 does not protrude from the opening 330 of the end cap 328, or is otherwise flush with the exterior surface of the end cap 328.

The cap 336 and tip 338 are secured within the sleeve fitting 324 and the end cap 328 by first filling the upper portion of the cap 328 with an epoxy or thermosetting material 342 and thereafter press fitting the cap 336 and tip 338 therewithin. The tip 338 is received within the opening 330 and the cap 336 is secured within the upper end of the interior of the end cap 328. The sleeve 324, cap 336, tip 338, and end cap 328 cooperatively define the enema device 310. The enema device 310 replaces the standard cap 36 and nozzle 38 that comes attached to the bottle 32 of the prepackaged enema 12. It should be appreciated that the enema device 310 may be instead made in any other suitable manner.

To use the enema device 310, the patient first removes the cap 36 that is attached to the bottle 32 of the prepackaged enema 12. The patient then secures the enema device 310 to the bottle 32 by inserting the threaded portion of the bottle neck 34 into the cap 336 and thereafter twisting the end cap 328. The patient then positions the enema device 310 such that the end cap 322 abuts the buttocks and the opening 330 is aligned with the rectum. While holding the bottle 32 and enema device 310 in this position, the patient then squeezes the bottle 32 to force the medicated liquid out of the one-way valve 40, through the tip 338, and outwardly through the opening 330 of the enema device 310 into the patient's rectum. Although the enema device 310 can be used to self-administer the enema, it is preferred that another person positions the enema device 310 and administers the enema to the patient.

While the preferred embodiment has been illustrated and described, it will be appreciated that various changes can be made therein without departing from the spirit and scope of the present disclosure.

The embodiments of the present disclosure in which an exclusive property or privilege is claimed are defined as follows:

1. An enema device for use with a source of enema fluid, the enema device comprising:
(a) a hollow elongated body having first and second ends and an interior extending between the first and second ends;
(b) a feed portion formed at the first end of the hollow elongated body and in fluid communication with the interior of the hollow elongated body, the feed portion adapted to be placed into fluid communication with a source of enema fluid;
(c) an applicator portion disposed at the second end of the elongated body, the applicator portion defining a discharge opening in communication with the interior of the hollow elongated body.

2. The enema device of claim 1, wherein the hollow elongated body is rigid such that it is adapted to pivot against an abutment device.

3. The enema device of claim 1, wherein the applicator portion includes an end face that is contoured to engage a patient's buttocks and align the discharge opening with the patient's rectum.

4. The enema device of claim 3, wherein the applicator portion includes a self-centering protrusion formed on the end face around the discharge opening.

5. The enema device of claim 3, wherein the end face of the applicator portion is convex-shaped.

6. The enema device of claim 3, wherein the end face of the applicator portion is substantially flat.

7. The enema device of claim 1, wherein the feed portion comprises a tube extending from the first end of the hollow elongated body through the interior of the hollow elongated body.

8. The enema device of claim 7, wherein the tube is adapted to communicate with a nozzle of the source of enema fluid.

9. The enema device of claim 8, wherein the tube extending along the interior of the elongated hollow body is received within the discharge opening of the applicator portion.

10. The enema device of claim 1, wherein the first end of the hollow elongated body has portions defining a tapered opening for receiving a nozzle of the source of enema fluid.

11. An enema device for administering a prepackaged enema to a patient in a sitting position, the enema device comprising:
(a) a hollow elongated body having an interior, the hollow elongated body being rigid such that it is adapted to pivot against an abutment device;
(b) a feed portion formed at an upper end of the elongated body, the feed portion including a first opening in communication with the interior of the hollow elongated body and adapted to be placed into fluid communication with a prepackaged enema containing medicated fluid; and
(c) an applicator portion disposed at a lower end of the elongated body, the applicator portion defining a second opening in communication with the interior of the hollow elongated body, wherein the applicator portion is formed at an angle from the hollow elongated body such that it engages a predetermined portion of a patient's body when the elongated body is pivoted on the portion of the abutment device.

12. The enema device of claim 11, wherein the applicator portion includes an end face that is contoured to engage a patient's buttocks and align the opening with the patient's rectum.

13. The enema device of claim 12, wherein the applicator portion includes a self-centering protrusion formed on the end face around the second opening.

14. The enema device of claim 12, wherein the end face of the applicator portion is convex-shaped.

15. The enema device of claim 11, wherein the feed portion includes a tube extending from the first opening in the upper end of the hollow elongated body and through the interior of the hollow elongated body.

16. The enema device of claim 15, wherein the tube is adapted to communicate with a nozzle of the prepackaged enema.

17. The enema device of claim 16, wherein the tube extends along the interior of the elongated hollow body and is received within the second opening of the applicator portion.

18. An enema device for use with a prepackaged enema containing medicated fluid, the enema device comprising:
(a) a cap releasably securable to a prepackaged enema;
(b) a nozzle disposed on the cap, the nozzle in fluid communication with the prepackaged enema when the cap is secured to the prepackaged enema;
(c) an end cap having an end face and a discharge opening formed therein, wherein the cap and nozzle are received within the end cap such that the nozzle is disposed within the opening.

19. The enema device of claim 18, wherein the nozzle is substantially flush with the end face of the end cap.

20. The enema device of claim 18, wherein the end face is contoured to engage a patient's buttocks and align the opening with the patient's rectum.
